(19) United States
(12) Patent Application Publication
(10) Pub. No.: US 2023/0389894 A1
(43) Pub. Date: Dec. 7, 2023

(54) IMAGE DIAGNOSIS CATHETER

(71) Applicant: TERUMO KABUSHIKI KAISHA, Tokyo (JP)

(72) Inventors: Tomohiro FUKUDA, Hadano-shi (JP); Soichiro SUGIHARA, Fujinomiya-shi (JP); Yoichi ITO, Fujinomiya-shi (JP)

(73) Assignee: TERUMO KABUSHIKI KAISHA, Tokyo (JP)

(21) Appl. No.: 18/454,146

(22) Filed: Aug. 23, 2023

Related U.S. Application Data
(63) Continuation of application No. PCT/JP2022/005434, filed on Feb. 10, 2022.

(30) Foreign Application Priority Data
Feb. 26, 2021 (JP) 2021-031024

(51) Int. Cl. A61B 8/00; A61B 8/12; A61B 5/00; A61B 8/08

(52) U.S. Cl. CPC A61B 8/445; A61B 8/12; A61B 5/0066; A61B 5/6852; A61B 5/0035; A61B 8/5238

(57) ABSTRACT

An image diagnosis catheter includes: an outer tube; a support tube provided radially inside of the outer tube; a spacer integrally connecting the outer tube with the support tube; and an inner tube provided radially inside of the outer tube and radially outside of the support tube and movable relative to the outer tube and the support tube in an axial direction, in which a communication passage causing a flow passage between the outer tube and the support tube to communicate with an inside of the support tube is formed by the spacer.

### CROSS-REFERENCES TO RELATED APPLICATIONS

This application is a continuation of International Application No. PCT/JP2022/005434 filed on Feb. 10, 2022, which claims priority to Japanese Application No. 2021-0314024 filed on Feb. 26, 2021, the entire content of both of which is incorporated herein by reference.

### TECHNOLOGICAL FIELD

The present disclosure generally relates to an image diagnosis catheter.

### BACKGROUND DISCUSSION

The image diagnosis catheter generally has a pull-back mechanism that changes a relative position between a sheath and a drive shaft on a side close to a user in order to continuously observe a body cavity cross section (see, for example, Japanese Patent Application Publication No. 2002-360578 A). The pull-back mechanism includes an outer tube, a support tube provided radially inside the outer tube and radially outside the drive shaft, a spacer integrally connecting the outer tube with the support tube, and an inner tube provided radially inside the outer tube and radially outside the support tube and movable relative to the outer tube and the support tube in an axial direction.

When the image diagnosis catheter is used, priming is performed to fill a lumen of the image diagnosis catheter with a liquid. The priming is usually performed in a state in which the inner tube is drawn out from the outer tube.

The image diagnosis catheter in the related art has a structure in which a flow passage between an outer tube and a support tube is closed by a spacer and communicates with the inside of the support tube through a notch such as a hole or a slit provided on the support tube in the vicinity of a closed portion. For this reason, there are problems that a flow passage resistance in the pull-back mechanism is relatively large and air in the pull-back mechanism can be difficult to remove.

### SUMMARY

An image diagnosis catheter is disclosed that is capable of implementing satisfactory priming in a pull-back mechanism.

According to an aspect of the present disclosure, an image diagnosis catheter is disclosed that includes: an outer tube; a support tube provided radially inside the outer tube; a spacer integrally connecting the outer tube with the support tube; and an inner tube provided radially inside of the outer tube and radially outside of the support tube and movable relative to the outer tube and the support tube in an axial direction, in which a communication passage causing a flow passage between the outer tube and the support tube to communicate with an inside of the support tube is formed by the spacer.

According to an embodiment of the present disclosure, the image diagnosis catheter further includes a connector joined to the outer tube, in which the spacer is joined to the support tube, and the spacer has a stopper positioned between the outer tube and the connector in the axial direction.

According to an embodiment of the present disclosure, the communication passage is defined by the spacer, the outer tube, and the connector.

According to an embodiment of the present disclosure, the spacer includes a spacer body positioned between an outer circumferential surface of the support tube and an inner circumferential surface of the outer tube, and a protruding portion protruding from a distal end of the spacer body toward the distal end on the distal end side with respect to a distal end surface of the outer tube.

According to an embodiment of the present disclosure, the spacer body has a cylindrical shape, and an outer circumferential surface of the spacer body has two portions having a flat surface shape, the two portions being separated from each other in a first direction along a radial direction and extending in the axial direction.

In accordance with another aspect, an image diagnosis catheter includes: an outer tube; a support tube provided radially inside of the outer tube; a spacer configured to connect the outer tube with the support tube, and the spacer configured to form a flow passage between the outer tube and the support tube, the flow passage configured to be in communication with an inside of the support tube; and wherein the spacer includes a spacer body positioned between an outer circumferential surface of the support tube and an inner circumferential surface of the outer tube, and a protruding portion protruding from a distal end of the spacer body toward a distal end on a distal end side of the image diagnosis catheter with respect to a distal end surface of the outer tube.

In accordance with a further aspect, a spacer configured to connect an outer tube with a support tube of an image diagnosis catheter is disclosed, the spacer including: a spacer body positioned between an outer circumferential surface of the support tube and an inner circumferential surface of the outer tube; and a protruding portion protruding distally from a distal end of the spacer body.

According to the present disclosure, there can be provided an image diagnosis catheter capable of implementing satisfactory priming in a pull-back mechanism.

### BRIEF DESCRIPTION OF THE DRAWINGS

FIG. 1 is a plan view illustrating a state in which an external device is connected to an image diagnosis catheter according to a first embodiment.

FIG. 2A is a side view illustrating the image diagnosis catheter illustrated in FIG. 1 in a state before a pull-back operation.

FIG. 2B is a side view illustrating the image diagnosis catheter illustrated in FIG. 1 in a state after a pull-back operation.

FIG. 3 is a cross-sectional view illustrating a distal end of the image diagnosis catheter illustrated in FIG. 1.

FIG. 4 is a cross-sectional view illustrating a proximal end of the image diagnosis catheter illustrated in FIG. 1.

FIG. 5A is a cross-sectional view illustrating a part of a pull-back mechanism of the image diagnosis catheter illustrated in FIG. 1.

FIG. 5B is a cross-sectional view of the pull-back mechanism illustrated in FIG. 5A when viewed in a direction different by 90°.

FIG. 6A is a perspective view illustrating an outer tube, a spacer, and a support tube, which are illustrated in FIG. 5A.

FIG. 6B is a plan view when viewed from a distal end side of the outer tube, spacer, and support tube, which are illustrated in FIG. 6A.

FIG. 7 is a cross-sectional view illustrating a part of a pull-back mechanism according to a second embodiment.

FIG. 8A is a perspective view illustrating an outer tube, a spacer, and a support tube, which are illustrated in FIG. 7.

FIG. 8B is a plan view when viewed from a distal end side of the outer tube, spacer, and support tube, which are illustrated in FIG. 8A.

FIG. 9A is a perspective view illustrating an outer tube, a spacer, and a support tube according to a third embodiment.

FIG. 9B is a plan view when viewed from a distal end side of the outer tube, spacer, and support tube, which are illustrated in FIG. 9A.

FIG. 10A is a perspective view illustrating an outer tube, a spacer, and a support tube according to a fourth embodiment.

FIG. 10B is a plan view when viewed from a distal end side of the outer tube, spacer, and support tube, which are illustrated in FIG. 10A.

FIG. 11 is a perspective view illustrating an outer tube, a spacer, and a support tube according to a fifth embodiment.

### DETAILED DESCRIPTION

Hereinafter, embodiments of an image diagnosis catheter according to the present disclosure will be described in detail by giving examples with reference to the drawings.

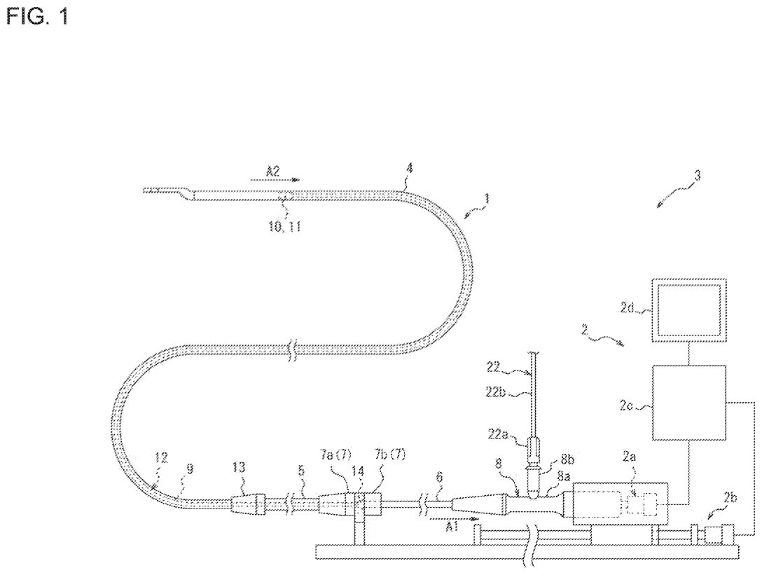

An image diagnosis catheter 1 according to the present embodiment is a dual-type image diagnosis catheter that uses both an intravascular ultrasound (IVUS) diagnosis method and an optical coherence tomography (OCT) diagnosis method. In the dual-type image diagnosis catheter 1, there are three types of modes including a mode of acquiring a tomographic image obtained only by IVUS, a mode of acquiring a tomographic image obtained only by OCT, and a mode of acquiring a tomographic image obtained by IVUS and OCT, and these modes can be used in a switching manner. As illustrated in FIG. 1, the image diagnosis catheter 1 is connected to an external device 2 and driven. The image diagnosis catheter 1 and the external device 2 constitute an image diagnosis apparatus 3.

As illustrated in FIGS. 1 to 4, the image diagnosis catheter 1 can include: a sheath 4 to be inserted into a body cavity such as a vessel (blood vessel such as a coronary artery) of a living body; an outer tube 5 connected to a proximal end of the sheath 4; an inner tube 6 to be inserted into the outer tube 5 so that the inner tube 6 is movable forward and backward (i.e., proximally and distally); a unit connector 7 connected to a proximal end of the outer tube 5, the unit connector 7 holding the inner tube 6 so that the inner tube 6 is movable forward and backward, and the unit connector 7 is capable of releasing the holding of the inner tube 6; and a hub 8 connected to a proximal end of the inner tube 6. Furthermore, the image diagnosis catheter 1 further includes an imaging core 12 including a drive shaft 9, a housing 10 fixed to a distal end of the drive shaft 9, and a signal transmission and reception unit 11 that is accommodated in the housing 10 and transmits and receives a signal that is an ultrasound wave and/or light. The imaging core 12 is inserted into the sheath 4, the outer tube 5, and the inner tube 6, and the imaging core 12 can move forward and backward in an axial direction integrally with the inner tube 6 with respect to the sheath 4 and the outer tube 5.

In the present specification, the distal end means an end on a side of the image diagnosis catheter 1 to be inserted into a body cavity, the proximal end means an end on a side of the image diagnosis catheter 1 to be held outside the body cavity, the axial direction means a direction along a central axis O of the drive shaft 9 (that is, an extending direction of the drive shaft 9), a radial direction means a direction along a straight line orthogonal to the central axis O, and a circumferential direction means a direction around the central axis O.

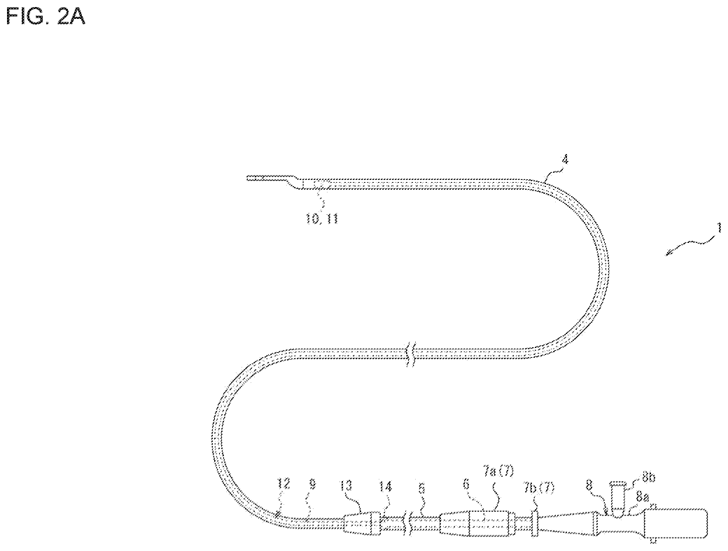

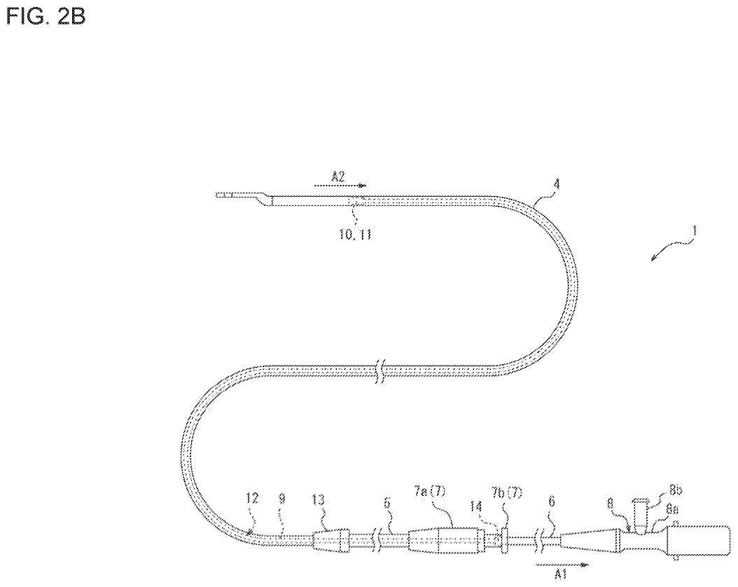

As illustrated in FIG. 2A, the drive shaft 9 extends to the inside of the hub 8 through the sheath 4, the outer tube 5, and the inner tube 6. The hub 8, the inner tube 6, the drive shaft 9, the housing 10, and the signal transmission and reception unit 11 are connected to each other so as to be movable forward and backward in the axial direction integrally with respect to the sheath 4 and the outer tube 5. Therefore, for example, when an operation of pushing the hub 8 toward the distal end side, that is, a push-in operation is performed, the inner tube 6 connected to the hub 8 is pushed into the outer tube 5 and the unit connector 7, and the drive shaft 9, the housing 10, and the signal transmission and reception unit 11. Specifically, the imaging core 12 that includes the drive shaft 9, the housing 10, and the signal transmission and reception unit 11 moves forward inside the sheath 4, that is, toward the distal end side. For example, when an operation of pulling the hub 8 toward the proximal end side, that is, a pull-back operation is performed, the inner tube 6 is drawn out from the outer tube 5 and the unit connector 7 as indicated by an arrow A1 in FIGS. 1 and 2B, and the imaging core 12 moves toward the proximal end side inside the sheath 4 as indicated by an arrow A2.

As illustrated in FIG. 2A, when the inner tube 6 is pushed toward the distal end side, a distal end of the inner tube 6 reaches the vicinity of a relay connector 13. At this time, the signal transmission and reception unit 11 is located at a distal end of the sheath 4 (near a distal end surface of a lumen of the sheath 4). The relay connector 13 connects the sheath 4 with the outer tube 5.

As illustrated in FIG. 2B, a locking portion 14 for helping prevent disengagement of the inner tube 6 from the outer tube 5 is provided at the distal end of the inner tube 6. Specifically, the locking portion 14 helps prevent the inner tube 6 from coming out of the outer tube 5. Furthermore, the unit connector 7 includes a distal-end-side portion connector 7a and a proximal-end-side portion connector 7b detachably connected to the distal-end-side portion connector 7a. The locking portion 14 is configured to be hooked at a predetermined position on an inner wall of the proximal-end-side portion connector 7b of the unit connector 7 when the hub 8 is pulled to the proximal end side, that is, when the inner tube 6 is pulled out from the outer tube 5 and the unit connector 7. As the proximal-end-side portion connector 7b is detached from the distal-end-side portion connector 7a, the inner tube 6 including the locking portion 14 can come out of the outer tube 5.

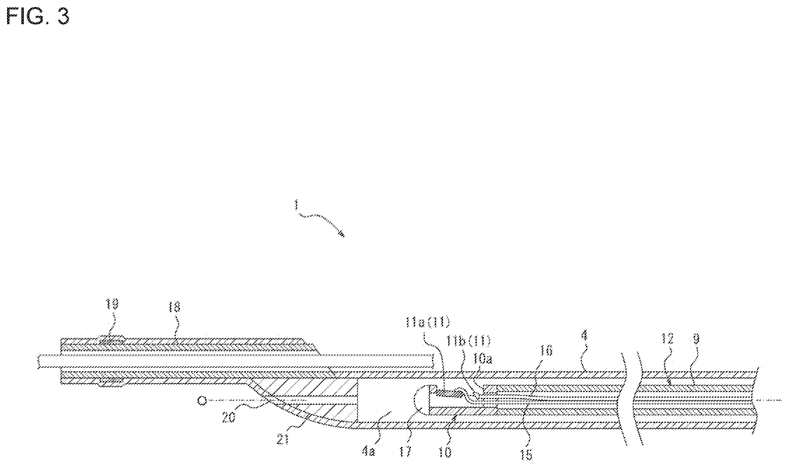

As illustrated in FIG. 3, the drive shaft 9 is an elongated hollow member, which houses an electric signal line (electric cable) 15 and an optical signal line (optical fiber) 16, which are connected to the signal transmission and reception unit 11. The electrical signal line (electric cable) 15 and the optical signal line (optical fiber) 16 are disposed inside the drive shaft 9.

The drive shaft 9 can be formed by a coil shaft. The coil shaft can be formed using, for example, multi-layer coils having different winding directions. Each of the coils can be a multi-wire winding type. The coil shaft can be formed, for example, using three-layer coils of a double winding type, the number of layers and the number of wires can be appropriately changed. Each of the coils can be made of, for example, metal such as stainless steel or a nickel-titanium (Ni—Ti) alloy.

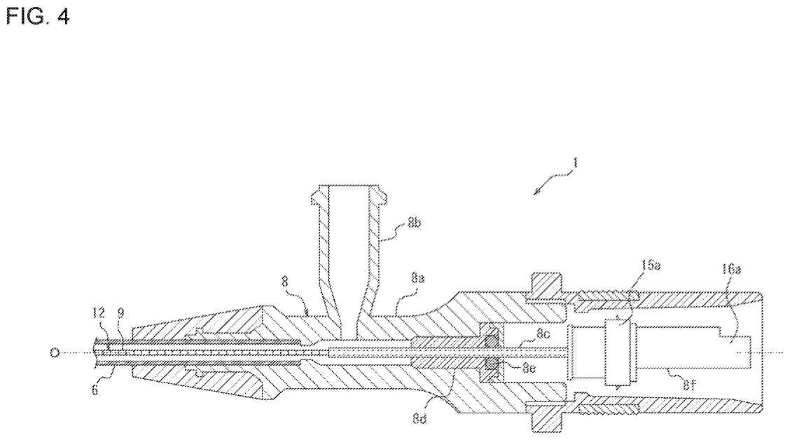

The signal transmission and reception unit 11 includes an ultrasound transmission and reception unit 11a that transmits and receives an ultrasound wave and an optical transmission and reception unit 11b that transmits and receives light. The ultrasound transmission and reception unit 11a includes a transducer that transmits an ultrasound wave based on a pulse signal into a body cavity and receives an ultrasound wave reflected from a biological tissue in the body cavity. The transducer is electrically connected to an electrical connector 15a (see FIG. 4) via the electric signal line 15. The transducer can be made of, for example, a piezoelectric material such as ceramic or quartz.

The optical transmission and reception unit 11b includes an optical element that transmits light into a body cavity and receives light reflected from a biological tissue in the body cavity. The optical element is optically connected to an optical connector 16a (see FIG. 4) via the optical signal line 16. The optical element can be formed using, for example, a lens such as a ball lens.

The signal transmission and reception unit 11 is accommodated in the housing 10. A proximal end of the housing 10 is fixed to the distal end of the drive shaft 9. The housing 10 can be, for example, formed using a cylindrical tube made of metal, and can be provided with an opening 10a on a circumferential surface of the housing 10 so as not to hinder the progress of a signal transmitted and received by the signal transmission and reception unit 11. The housing 10 can be formed by, for example, laser processing or the like. The housing 10 may be formed by shaving a metal lump (or a block of metal), metal injection molding (MIM), or the like.

A distal end member 17 is provided at a distal end of the housing 10. The distal end member 17 can have a substantially hemispherical outer shape, and accordingly, can suppress friction and catching with an inner surface of the sheath 4. Note that the distal end member 17 may not be provided.

The sheath 4 has a lumen 4a into which the drive shaft 9 is inserted so that the drive shaft 9 is movable forward and backward. A tubular guide wire insertion member 18 through which a guide wire can pass is attached to the distal end of the sheath 4, which shifts a lumen for the guide wire from an axial center of the lumen of the sheath 4. The sheath 4 and the guide wire insertion member 18 are bonded by welding or the like. The guide wire insertion member 18 can be provided with a marker 19 having an radiopaque property. The marker 19 can be, for example, a tubular member made of metal having a relatively high X-ray impermeability such as platinum (Pt) or gold (Au).

A communication hole 20 that communicates with the inside and the outside of the lumen 4a is formed at the distal end of the sheath 4. Furthermore, a reinforcing member 21 joined to the guide wire insertion member 18 can be provided at a distal end of the lumen 4a of the sheath 4. The reinforcing member 21 has a through-hole formed to allow communication between the communication hole 20 and the inside of the lumen 4a disposed on the proximal end side with respect to the reinforcing member 21. The reinforcing member 21 may not be provided at the distal end of the sheath 4.

The communication hole 20 is a priming solution discharge hole for discharging a priming solution. When the image diagnosis catheter 1 is used, the priming solution can be released from the communication hole 20 to the outside to discharge a gas such as air from the inside of the sheath 4 together with the priming solution at the time of performing priming processing of filling the inside of the sheath 4 with the priming solution.

A distal-end-side portion of the sheath 4, which is a range in which the signal transmission and reception unit 11 moves in the axial direction of the sheath 4, forms a window portion having a higher signal transmission property than other portions. The sheath 4, the guide wire insertion member 18, and the reinforcing member 21 are made of a flexible material, and the material is not particularly limited. Examples of the materials of the sheath 4, the guide wire insertion member 18, and the reinforcing member 21 can include various thermoplastic elastomers such as a styrene-based material, a polyolefin-based material, a polyurethane-based material, a polyester-based material, a polyamide-based material, a polyimide-based material, a polybutadiene-based material, a transpolyisoprene-based material, a fluororubber-based material, and a chlorinated polyethylene-based material, and one or a combination of two or more of these (a polymer alloy, a polymer blend, a laminate, or the like) can also be used.

As illustrated in FIG. 4, the hub 8 can include: a hub body 8a having a tubular shape coaxial with the inner tube 6 and integrally attached to the external device 2 in a detachable manner; a port 8b protruding radially outward from the hub body 8a and communicating with the inside of the hub body 8a; a connection pipe 8c integrally attached to an outer circumferential surface of the drive shaft 9; a bearing 8d rotatably supporting the connection pipe 8c; a seal member 8e configured to help prevent the priming solution from leaking from a gap between the connection pipe 8c and the bearing 8d toward the proximal end side; and a connector portion 8f provided with the electrical connector 15a and the optical connector 16a and integrally attached to a first drive unit 2a of the external device 2 in a detachable manner. The connector portion 8f can be rotatable integrally with the connection pipe 8c and the drive shaft 9.

The proximal end of the inner tube 6 is integrally connected to a distal end of the hub body 8a. The drive shaft 9 is extends from the inner tube 6 inside the hub body 8a.

As illustrated in FIG. 1, an injection device 22 (see FIG. 1) that injects the priming solution at the time of performing the priming processing is connected to the port 8b. The injection device 22 includes a connector 22a connected to the port 8b and a syringe connected to the connector 22a via a tube 22b.

The external device 2 includes the first drive unit 2a for rotationally driving the drive shaft 9 and a second drive unit 2b for moving the drive shaft 9 in the axial direction (that is, for push-in operation and pull-back operation). The first drive unit 2a can be configured by, for example, an electric motor. The second drive unit 2b can be configured by, for example, an electric motor and a direct motion conversion mechanism. The direct motion conversion mechanism can convert rotational motion into linear motion, and can be configured by, for example, a ball screw, a rack-and-pinion mechanism, or the like.

Operation of the first drive unit 2a and operation of the second drive unit 2b are controlled by a control device 2c electrically connected to the first drive unit 2a and the second drive unit 2b. The control device 2c includes a central processing unit (CPU) and a memory. The control device 2c is electrically connected to a display 2d.

A signal received by the ultrasound transmission and reception unit 11a is transmitted to the control device 2c via the electrical connector 15a, subjected to predetermined processing, and displayed as an image on the display 2d. A signal received by the optical transmission and reception unit 11b is transmitted to the control device 2c via the optical connector 16a, subjected to predetermined processing, and displayed as an image on the display 2d.

At the time of diagnosis, the imaging core 12 moves backward at a constant speed inside the lumen 4a of the sheath 4 by the pull-back operation by the second drive unit 2b of the external device 2 in a state in which the sheath 4 is inserted into the body cavity and the imaging core 12 is rotationally driven at a constant rotational speed of about 1000 rpm (revolutions per minute) to 10000 rpm by the first drive unit 2a of the external device 2. At this time, the control device 2c of the external device 2 causes the signal transmission and reception unit 11 to transmit and receive a signal. A state of a tissue around the body cavity is displayed as an image on the display 2d based on the signal received by scanning operation performed by rotating and moving the imaging core 12 backward.

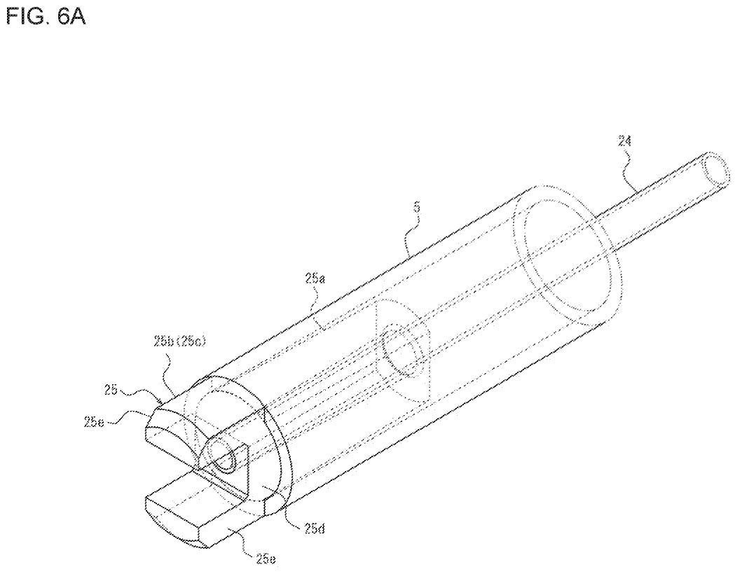

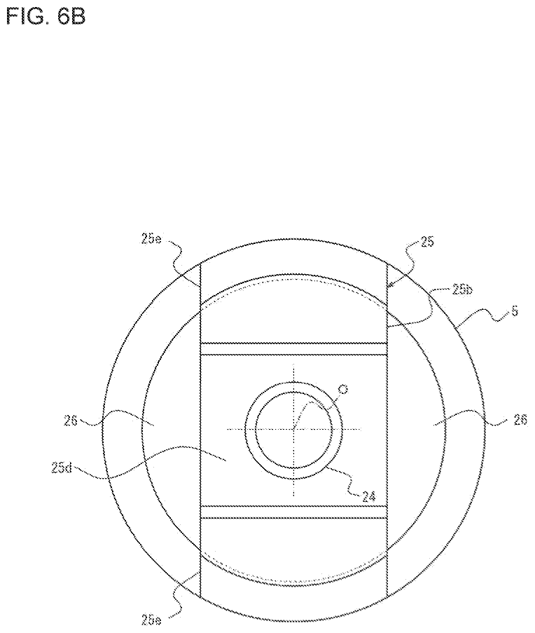

As described above, the image diagnosis catheter 1 has the pull-back mechanism 23 that changes a relative position between the sheath 4 and the drive shaft 9 on a side close to a user in order to continuously observe a body cavity cross section. As described in FIGS. 2A and 2B, and FIGS. 5A to 6B, the pull-back mechanism 23 includes an outer tube 5, a support tube 24 provided radially inside the outer tube 5 and radially outside a drive shaft 9, a spacer 25 integrally connecting the outer tube 5 and the support tube 24, an inner tube 6 provided radially inside the outer tube 5 and radially outside the support tube 24 and movable relative to the outer tube 5 and the support tube 24 in an axial direction, a relay connector 13, and a unit connector 7. As described above, the relay connector 13 is integrally connected to the sheath 4, and the inner tube 6 is integrally connected to the hub 8.

In the pull-back mechanism 23, the outer tube 5, the support tube 24, the inner tube 6, and the drive shaft 9 are coaxially provided and have a common central axis O.

The relay connector 13 has a cylindrical shape, and has a proximal-end-side inner circumferential surface 13a having a columnar surface shape, and a distal-end-side inner circumferential surface 13c having a columnar surface shape connected to a distal end of the proximal end side inner circumferential surface 13a via an annular step portion 13b. An outer circumferential surface of the proximal end of the sheath 4 is joined to the distal-end-side inner circumferential surface 13c by welding or the like. An outer circumferential surface of the distal end of the outer tube 5 is joined to the proximal-end-side inner circumferential surface 13a by welding or the like.

The spacer 25 includes a spacer body 25a positioned between the outer circumferential surface of the support tube 24 and the inner circumferential surface of the outer tube 5, and a protruding portion 25b protruding from a distal end of the spacer body 25a toward the distal end on the distal end side with respect to the distal end surface of the outer tube 5. The protruding portion 25b constitutes a stopper 25c positioned between the outer tube 5 and the relay connector 13 in the axial direction. The stopper 25c has a proximal-end-side end surface whose movement toward the proximal end side is restricted by coming into contact with the distal end surface of the outer tube 5, and a distal-end-side end surface whose movement toward the distal end side is restricted by coming into contact with the step portion 13b of the relay connector 13. The spacer 25 can be made of, for example, synthetic resin or metal.

The spacer body 25a has a cylindrical shape, and the outer circumferential surface of the spacer body 25a has two portions having a flat surface shape, the two portions being separated from each other in a first direction along a radial direction and extending in the axial direction. Therefore, two axial flow passages 26 extending in the axial direction respectively at two portions separated from each other in the first direction are formed between the outer circumferential surface of the spacer body 25a and the inner circumferential surface of the outer tube 5.

The protruding portion 25b includes a base portion 25d integrally connected to the distal end of the spacer body 25a, and two protruding pieces 25e extending from a distal end of the base portion 25d toward the distal end on the distal end side with respect to a distal end of the support tube 24. Two protruding pieces 25e are separated from each other in a second direction along the radial direction and perpendicular to the first direction. An outer circumferential surface of the protruding portion 25b has two portions having a flat surface parallel to the central axis O, the two portions being separated from each other in the first direction. Therefore, two gaps 27 separated from each other in the first direction are formed between the outer circumferential surface of the protruding portion 25b and the proximal-end-side inner circumferential surface 13a of the relay connector 13. Furthermore, a gap 28 is formed between two protruding pieces 25e. The distal end of each of the axial flow passages 26 communicates with the distal end of the lumen of the support tube 24 via one gap 27 and the gap 28.

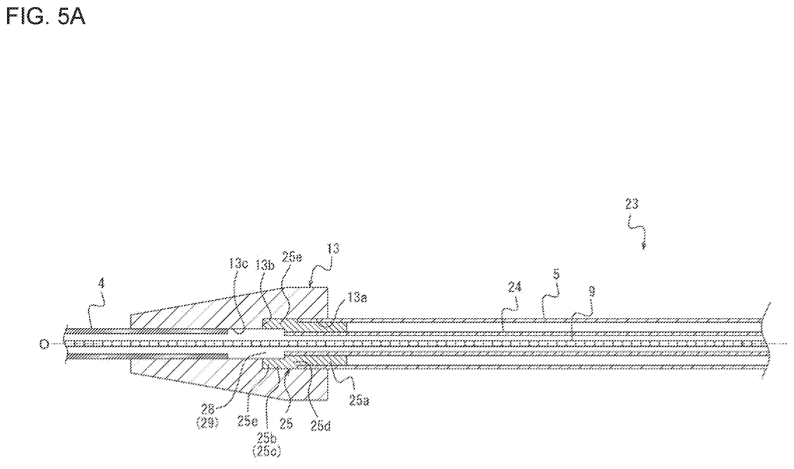

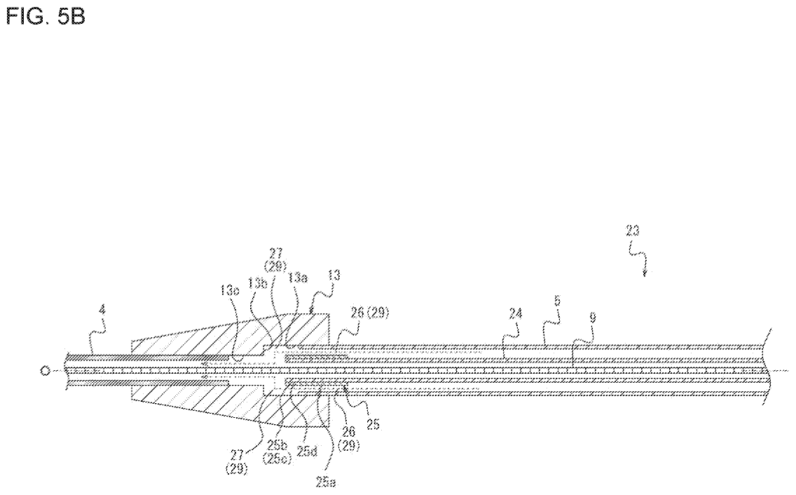

As described above, two axial flow passages 26, two gaps 27, and the gap 28 constitute a communication passage (or communication path) 29 (see FIGS. 5A to 5B) that causes the flow passage between the outer tube 5 and the support tube 24 to communicate with the inside of the support tube 24. The communication passage 29 is defined by the spacer 25, the outer tube 5, and the relay connector 13.

The outer circumferential surface of the distal end of the support tube 24 is joined to the inner circumferential surface of the spacer body 25a by welding or the like. The outer tube 5 and the spacer 25 are not joined. As described above, since the outer tube 5 is joined to the relay connector 13, the spacer 25 is joined to the support tube 24, and the spacer 25 has the stopper 25c, the relay connector 13, the outer tube 5, the support tube 24, and the spacer 25 can be efficiently integrated by a small number of joining portions. However, the connection strength of the pull-back mechanism 23 may be further increased by joining the outer tube 5 and the spacer 25 by welding or the like.

The support tube 24 can be formed of, for example, a single layer of a coil or a tube, or a plurality of layers of coils or tubes. The support tube 24 can be made of, for example, synthetic resin or metal. When the inner tube 6 and the drive shaft 9 are moved forward with respect to the outer tube 5 by the push-in operation, the drive shaft 9 is supported by the support tube 24 from the radially outer side. Therefore, it is possible to prevent the drive shaft 9 from being buckled in the outer tube 5, which can hinder the relatively smooth forward-movement of the drive shaft 9.

The priming is usually performed in a state in which the inner tube 6 is drawn out (i.e., extends) from the outer tube 5 (see FIG. 1). At the time of priming, the priming solution introduced from the port 8b passes through the inner tube 6, and branches into a flow passage between the outer tube 5 and the support tube 24 and a flow passage inside the support tube 24 to flow toward the distal end side. As indicated by a dashed line arrow in FIG. 5B, the priming solution flowing through the flow passage between the outer tube 5 and the support tube 24 passes through the communication passage 29 formed by the spacer 25, joins a priming solution flowing through the flow passage inside the support tube 24, and flows toward the distal end side. As described above, since the communication passage 29 is formed by the spacer 25, the flow passage resistance in the pull-back mechanism 23 can be reduced, and the air in the pull-back mechanism 23 can be relatively easily released.

As described above, according to the present embodiment, since the communication passage 29 that causes the flow passage between the outer tube 5 and the support tube 24 to communicate with the inside of the support tube 24 is formed by the spacer 25, it is possible to realize satisfactory priming in the pull-back mechanism 23.

Furthermore, according to the present embodiment, since the relay connector 13 is joined to the outer tube 5, the spacer 25 is joined to the support tube 24, and the spacer 25 has the stopper 25c, the pull-back mechanism 23 that can be relatively easily assembled can be realized.

Furthermore, according to the present embodiment, since the communication passage 29 is defined by the spacer 25, the outer tube 5, and the relay connector 13, it is possible to more reliably realize satisfactory priming in the pull-back mechanism 23.

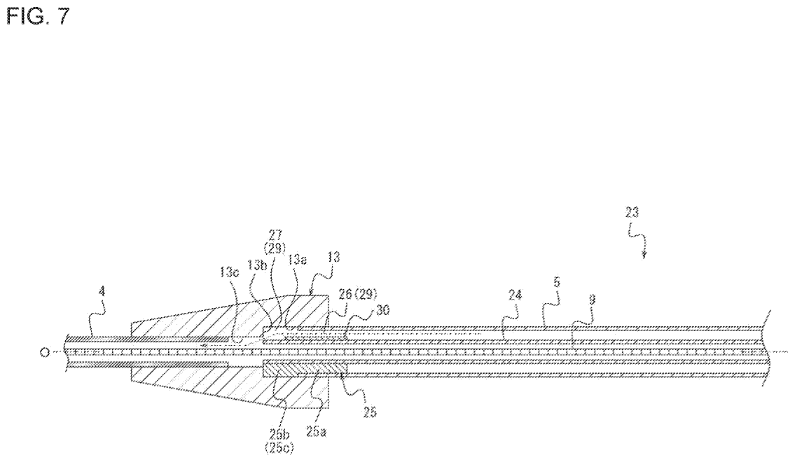

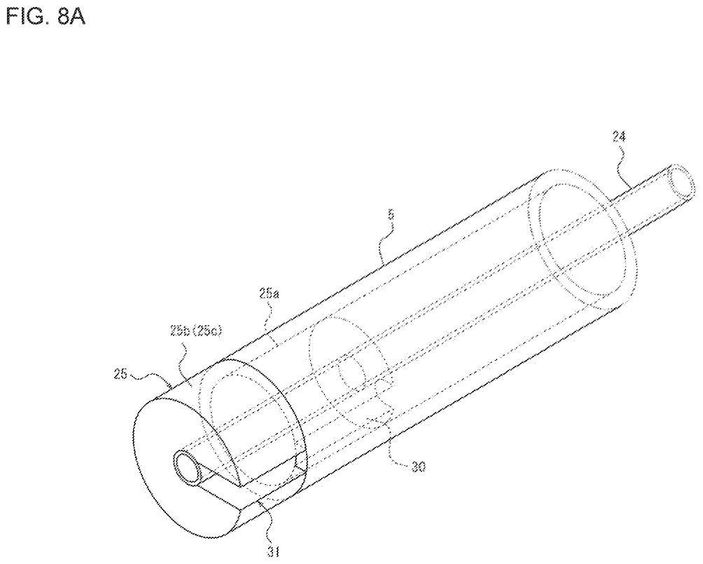

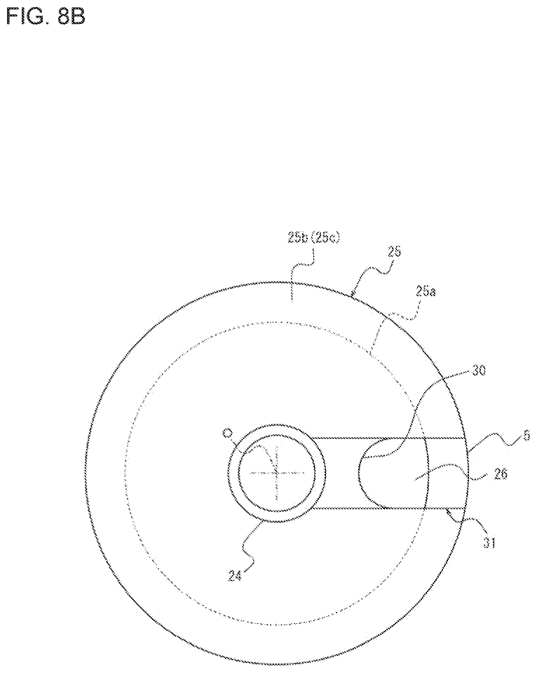

The configuration of the pull-back mechanism 23 can be changed as long as the communication passage 29 that causes the flow passage between the outer tube 5 and the support tube 24 to communicate with the inside of the support tube 24 is formed by the spacer 25. For example, the pull-back mechanism 23 may have a configuration as in a second embodiment illustrated in FIGS. 7 to 8B.

In the second embodiment, the spacer 25 has a spacer body 25a and a protruding portion 25b, and the protruding portion 25b constitutes a stopper 25c. In this point, the second embodiment has the same configuration as that of the first embodiment. However, in the second embodiment, the configuration of the spacer body 25a and the protruding portion 25b is different from that of the first embodiment.

In the second embodiment, the spacer body 25a has a cylindrical shape in which one axial groove 30 extending in the axial direction is provided at one position on the outer circumferential surface. Therefore, one axial flow passage 26 extending in the axial direction can be formed between the outer circumferential surface of the spacer body 25a and the inner circumferential surface of the outer tube 5.

Furthermore, the protruding portion 25b has a cylindrical shape, and one notch 31 extending in the axial direction and connected to the axial groove 30 is provided at one portion of the protruding portion 25b in the circumferential direction. The distal end surface of the protruding portion 25b is flush (i.e., even) with the distal end surface of the support tube 24. The notch 31 extends on the outer circumferential surface of the protruding portion 25b over the entire length in the axial direction, and radially extends on the distal end surface of the protruding portion 25b from the outer circumferential surface of the support tube 24 to an outer circumferential edge of the protruding portion 25b. Therefore, in the notch 31, one gap 27 is defined by the spacer 25, the support tube 24, and the relay connector 13.

Furthermore, the distal end surface of the protruding portion 25b is in contact with the step portion 13b of the relay connector 13, and the outer diameter of the proximal-end-side inner circumferential surface 13a of the relay connector 13 is larger than the outer diameter of the support tube 24. Therefore, the gap 27 causes the axial flow passage 26 to communicate with the distal end of the lumen of the support tube 24. As described above, in the second embodiment, the communication passage 29 is configured by one axial flow passage 26 and one gap 27. Other configurations are similar to those of the first embodiment. Even with such a configuration, satisfactory priming in the pull-back mechanism 23 can be realized.

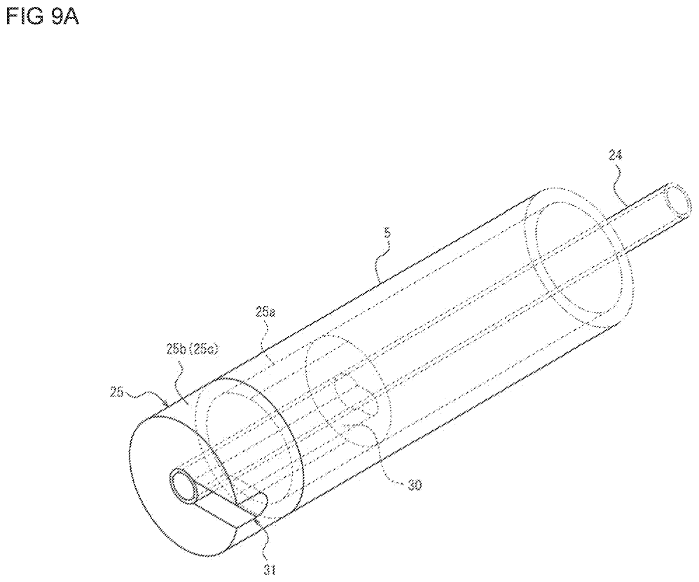

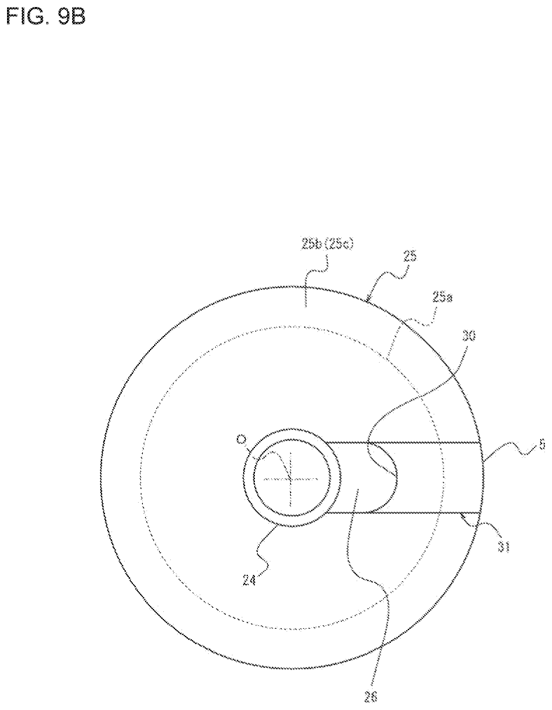

The pull-back mechanism 23 may have a configuration as in a third embodiment illustrated in FIGS. 9A and 9B. In the second embodiment, the axial flow passages 26 is defined by the axial groove 30 provided on the outer circumferential surface of the spacer body 25a and the inner circumferential surface of the outer tube 5, but in the third embodiment, the axial flow passage 26 is defined by the axial groove 30 provided on the inner circumferential surface of the spacer body 25a and the outer circumferential surface of the support tube 24. Furthermore, in the third embodiment, the notch 31 of the protruding portion 25b extends on the inner circumferential surface of the protruding portion 25b over the entire length in the axial direction, and radially extends on the distal end surface of the protruding portion 25b from the outer circumferential surface of the support tube 24 to an outer circumferential edge of the protruding portion 25b. Therefore, in the notch 31, one gap 27 is defined by the spacer 25, the support tube 24, and the relay connector 13. In the third embodiment, the communication passage 29 is configured by the axial flow passage 26 and the gap 27. Other configurations are similar to those of the second embodiment. With such a configuration, satisfactory priming in the pull-back mechanism 23 can be realized.

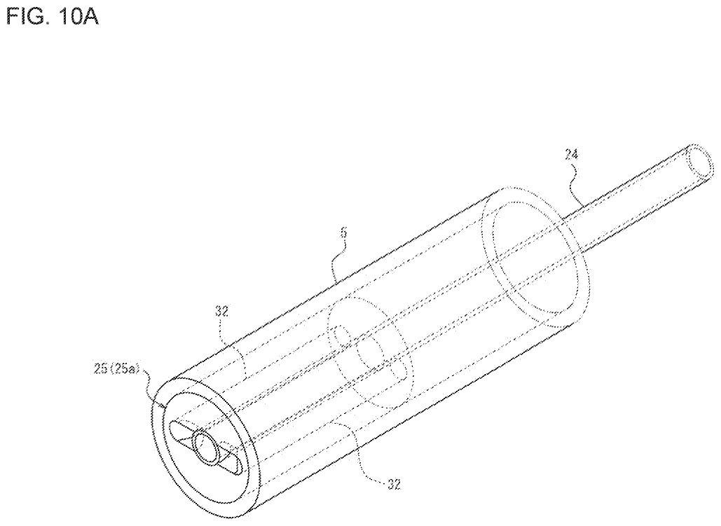

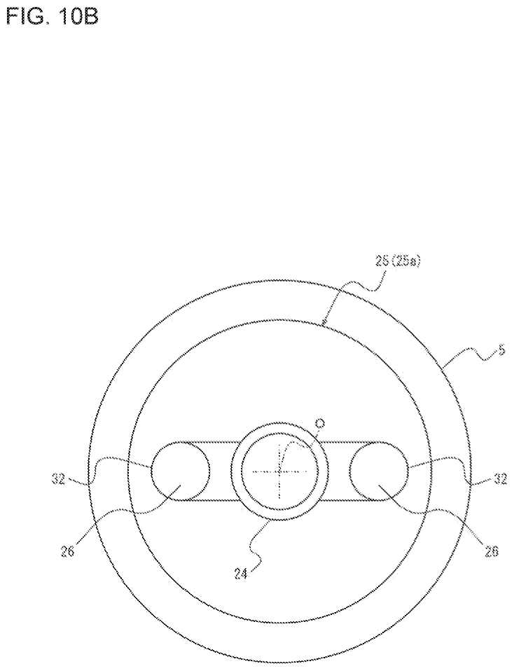

The pull-back mechanism 23 may have a configuration as in a fourth embodiment illustrated in FIGS. 10A and 10B. In the second embodiment, the axial flow passages 26 is defined by the axial groove 30 provided on the outer circumferential surface of the spacer body 25a and the inner circumferential surface of the outer tube 5, but in the fourth embodiment, the axial flow passages 26 is defined by two through-holes 32 axially penetrating the spacer body 25a, respectively at two portions in the circumferential direction. The distal end of each of the through-holes 32 extends radially inward to the outer circumferential surface of the support tube 24. Furthermore, in the fourth embodiment, the spacer 25 is not provided with the protruding portion 25b, and the spacer 25 is configured only by the spacer body 25a. Therefore, in the fourth embodiment, the communication passage 29 is configured by only two axial flow passages 26. Other configurations are similar to those of the second embodiment. With such a configuration, satisfactory priming in the pull-back mechanism 23 can be realized. Note that the number of the axial flow passages 26 can be appropriately increased or decreased.

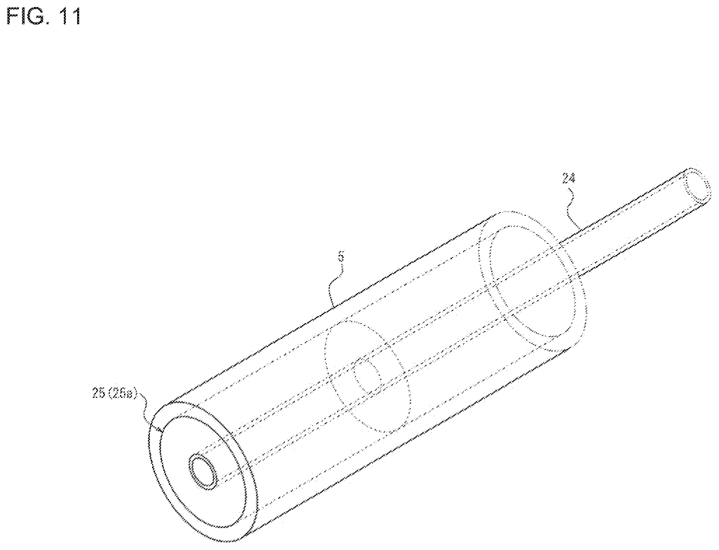

The pull-back mechanism 23 may have a configuration as in a fifth embodiment illustrated in FIG. 11. In the fourth embodiment, the axial flow passage 26 is defined by two through-holes 32 axially penetrating the spacer 25 (spacer body 25a), but in the fifth embodiment, the spacer 25 (spacer body 25a) is formed of a porous member, and the axial flow passage 26 is configured by a gap in the spacer 25. Therefore, in the fifth embodiment, the communication passage 29 is configured by only a gap in the spacer 25. Other configurations are similar to those of the fourth embodiment. With such a configuration, satisfactory priming in the pull-back mechanism 23 can be realized.

The above-described embodiment is merely an example of the present disclosure, and various modifications, for example, as described below can be made.

The image diagnosis catheter 1 can include an outer tube 5, a support tube 24 provided radially inside of the outer tube 5, a spacer 25 integrally connecting the outer tube 5 with the support tube 24, and an inner tube 6 provided radially inside of the outer tube 5 and radially outside of the support tube 24 and movable relative to the outer tube 5 and the support tube 24 in an axial direction, and can be changed as long as the communication passage 29 causing the flow passage between the outer tube 5 and the support tube 24 to communicate with the inside of the support tube 24 is formed by the spacer 25.

However, it is preferable that the image diagnosis catheter 1 includes a relay connector 13 joined to the outer tube 5, the spacer 25 is joined to the support tube 24, and the spacer 25 has the stopper 25c positioned between the outer tube 5 and the relay connector 13 in the axial direction.

Furthermore, the communication passage 29 is preferably defined by the spacer 25, the outer tube 5, and the relay connector 13.

Furthermore, the spacer 25 preferably includes a spacer body 25a positioned between the outer circumferential surface of the support tube 24 and the inner circumferential surface of the outer tube 5, and a protruding portion 25b protruding from a distal end of the spacer body 25a toward the distal end on the distal end side with respect to the distal end surface of the outer tube 5.

Furthermore, the spacer body 25a preferably has a cylindrical shape, and the outer circumferential surface of the spacer body 25a preferably has two portions having a flat surface shape, the two portions being separated from each other in a first direction along a radial direction and extending in the axial direction.

The image diagnosis catheter 1 is not limited to a dual-type image diagnosis catheter using both IVUS and OCT, and may be an image diagnosis catheter using only IVUS or only OCT.

The pull-back mechanism 23 is not limited to the configuration in which the relay connector 13 is integrally connected to the sheath 4 and the inner tube 6 is integrally connected to the hub 8, and may have a configuration in which the relay connector 13 is integrally connected to the hub 8 and the inner tube 6 is integrally connected to the sheath 4.

The detailed description above describes embodiments of an image diagnosis catheter. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An image diagnosis catheter comprising:
an outer tube;
a support tube provided radially inside of the outer tube;
a spacer configured to connect the outer tube with the support tube;
an inner tube provided radially inside of the outer tube and radially outside of the support tube, and the inner tube being configured to be movable relative to the outer tube and the support tube in an axial direction: and
wherein the spacer is configured to form a communication passage between the outer tube and the support tube, the communication passage configured to communicate with an inside of the support tube.

2. The image diagnosis catheter according to claim 1, further comprising:
a connector configured to be joined to the outer tube;
the spacer is configured to be joined to the support tube; and
the spacer has a stopper positioned between the outer tube and the connector in the axial direction.

3. The image diagnosis catheter according to claim 2, wherein the communication passage is defined by the spacer, the outer tube, and the connector.

4. The image diagnosis catheter according to claim 1, wherein the spacer includes a spacer body positioned between an outer circumferential surface of the support tube and an inner circumferential surface of the outer tube, and a protruding portion protruding from a distal end of the spacer body toward a distal end on a distal end side of the image diagnosis catheter with respect to a distal end surface of the outer tube.

5. The image diagnosis catheter according to claim 4, wherein the spacer body has a cylindrical shape.

6. The image diagnosis catheter according to claim 5, further comprising:
an outer circumferential surface of the spacer body has two portions having a flat surface shape, the two portions being separated from each other in a first direction along a radial direction and extending in the axial direction.

7. The image diagnosis catheter according to claim 1, further comprising:
a sheath configured to be inserted into a body cavity, the sheath being connected to a distal end of the outer tube; and
an imaging core, the imaging core including a drive shaft, a housing fixed to a distal end of the drive shaft, and a signal transmission and reception unit that is accommodated in the housing and configured to transmit and receive a signal that is one or more of an ultrasound wave and a light, and wherein the imaging core is configured to move forward and backward in an axial direction with the inner tube and with respect to the sheath and the outer tube.

8. The image diagnosis catheter according to claim 1, further comprising:
a drive shaft; and
a hub configured to be connected to the drive shaft, the hub including:
a hub body having a tubular shape coaxial with the inner tube and configured to be attachable to an external device;
a port protruding radially outward from the hub body and communicating with the inside of the hub body;
a connection pipe attached to an outer circumferential surface of the drive shaft;
a bearing rotatably supporting the connection pipe;
a seal member configured to prevent the priming solution from leaking from a gap between the connection pipe and the bearing toward a proximal end side of the hub; and
a connector portion provided with an electrical connector and an optical connector and configured to be attachable to the external device, and wherein the connector portion is configured to be rotatable with the connection pipe and the drive shaft.

9. The image diagnosis catheter according to claim 1, further comprising:
a locking portion provided at a distal end of the inner tube, the locking portion configured to prevent disengagement of the inner tube from the outer tube;
a connector, the connector includes a distal-end-side portion connector and a proximal-end-side portion connector detachably connected to the distal-end-side portion connector; and
wherein the locking portion is configured to be hooked at a predetermined position on an inner wall of the proximal-end-side portion connector of the connector when the inner tube is pulled out from the outer tube and the connector.

10. The image diagnosis catheter according to claim 4, wherein the spacer body has a cylindrical shape in which an axial groove extending in the axial direction is provided on the outer circumferential surface of the spacer body, the axial groove forming an axial flow passage extending in the axial direction between an outer circumferential surface of the spacer body and an inner circumferential surface of the outer tube.

11. The image diagnosis catheter according to claim 10, wherein the protruding portion has a cylindrical shape, and a notch extending in the axial direction and connected to the axial groove of the spacer body, the distal end surface of the protruding portion being flush with a distal end surface of the support tube and an axial flow passage is defined by the axial groove provided on the outer circumferential surface of the spacer body and the inner circumferential surface of the outer tube.

12. The image diagnosis catheter according to claim 11, wherein the axial flow passage is defined by the axial groove provided on the inner circumferential surface of the spacer body and the outer circumferential surface of the support tube, and the notch of the protruding portion extends on the inner circumferential surface of the protruding portion over the entire length in the axial direction, and radially extends on the distal end surface of the protruding portion from the outer circumferential surface of the support tube to an outer circumferential edge of the protruding portion.

13. The image diagnosis catheter according to claim 1, further comprising:
two or more through-holes axially penetrating a spacer body of the spacer, the two or more through-holes forming an axial flow passage, and wherein a distal end of each of the two or more through-holes extend radially inward to an outer circumferential surface of the support tube.

14. The image diagnosis catheter according to claim 1, further comprising:
two or more through-holes axially penetrating a spacer body of the spacer, and wherein the spacer body is formed of a porous member, and an axial flow passage is configured by a gap in the spacer.

15. An image diagnosis catheter comprising:
an outer tube;
a support tube provided radially inside of the outer tube;
a spacer configured to connect the outer tube with the support tube, and the spacer configured to form a flow passage between the outer tube and the support tube, the flow passage configured to be in communication with an inside of the support tube; and
wherein the spacer includes a spacer body positioned between an outer circumferential surface of the support tube and an inner circumferential surface of the outer tube, and a protruding portion protruding from a distal end of the spacer body toward a distal end on a distal end side of the image diagnosis catheter with respect to a distal end surface of the outer tube.

16. A spacer configured to connect an outer tube with a support tube of an image diagnosis catheter, the spacer comprising:
a spacer body positioned between an outer circumferential surface of the support tube and an inner circumferential surface of the outer tube; and
a protruding portion protruding distally from a distal end of the spacer body.

17. The spacer according to claim 16, further comprising:
an outer circumferential surface of the spacer body has two portions having a flat surface shape, the two portions being separated from each other in a first direction along a radial direction and extending in the axial direction; and
wherein the spacer body has a cylindrical shape, and an outer circumferential surface of the spacer body has two portions having a flat surface shape, the two portions being separated from each other in a first direction along a radial direction and extending in the axial direction.

18. The spacer according to claim 17, further comprising:
an axial groove extending in the axial direction on the outer circumferential surface of the spacer body, the axial groove forming an axial flow passage extending in the axial direction between an outer circumferential surface of the spacer body and an inner circumferential surface of the outer tube; and
wherein the protruding portion has a cylindrical shape, and a notch extending in the axial direction and connected to the axial groove of the spacer body, the distal end surface of the protruding portion being flush with a distal end surface of the support tube and an axial flow passage is defined by the axial groove provided on the outer circumferential surface of the spacer body and the inner circumferential surface of the outer tube.

19. The spacer according to claim 18, wherein the axial flow passage is defined by the axial groove provided on the inner circumferential surface of the spacer body and the outer circumferential surface of the support tube, and the notch of the protruding portion extends on the inner circumferential surface of the protruding portion over the entire length in the axial direction, and radially extends on the distal end surface of the protruding portion from the outer circumferential surface of the support tube to an outer circumferential edge of the protruding portion.

20. The spacer according to claim 1, further comprising:
two or more through-holes axially penetrating a spacer body of the spacer, the two or more through-holes forming an axial flow passage, and wherein a distal end of each of the two or more through-holes extend radially inward to an outer circumferential surface of the support tube.
